SEQUENCE LISTING (14 sequences)

SEQ ID NO: 1 - protein, 349 aa, artificial sequence
  RNA recognition domain
GRSRLLEDFRNNRYPNLQLREIAGHIMEFSQDQHGSRFIQLKLERATPAERQLVFNEILQAAYQLMVDVFGNYVIQKFFEFGSLEQKLALAERIRGHVLSLALQMYGCRVIQKALEFIPSDQQNEMVRELDGHVLKCVKDQNGNHVVQKCIECVQPQSLQFIIDAFKGQVFALSTHPYGCRVIQRILEHCLPDQTLPILEELHQHTEQLVQDQYGNYVIQHVLEHGRPEDKSKIVAEIRGNVLVLSQHKFASNVVEKCVTHASRTERAVLIDEVCTMNDGPHSALYTMMKDQYANYVVQKMIDVAEPGQRKIVMHKIRPHIATLRKYTYGKHILAKLEKYYMKNGVDLG

SEQ ID NO: 2 - protein, 429 aa, artificial sequence
  effector domain
KAERMGFTEVTPVTGASLRRTMLLLSRSPEAQPKTLPLTGSTFHDQIAMLSHRCFNTLTNSFQPSLLGRKILAAIIMKKDSEDMGVVVSLGTGNRCVKGDSLSLKGETVNDCHAEIISRRGFIRFLYSELMKYNSQTAKDSIFEPAKGGEKLQIKKTVSFHLYISTAPCGDGALFDKSCSDRAMESTESRHYPVFENPKQGKLRTKVENGEGTIPVESSDIVPTWDGIRLGERLRTMSCSDKILRWNVLGLQGALLTHFLQPIYLKSVTLGYLFSQGHLTRAICCRVTRDGSAFEDGLRHPFIVNHPKVGRVSIYDSKRQSGKTKETSVNWCLADGYDLEILDGTRGTVDGPRNELSRVSKKNIFLLFKKLCSFRYRRDLLRLSYGEAKKAARDYETAKNYFKKGLKDMGYGNWISKPQEEKNFYLCPV

SEQ ID NO: 3 - protein, 440 aa, artificial sequence
  the effector domain of ADAR2-isoform1
DQTPSRQPIPSEGLQLHLPQVLADAVSRLVLGKFGDLTDNFSSPHARRKVLAGVVMTTGTDVKDAKVISVSTGTKCINGEYMSDRGLALNDCHAEIISRRSLLRFLYTQLELYLNNKDDQKRSIFQKSERGGFRLKENVQFHLYISTSPCGDARIFSPHEPILEGSRSYTQAGVQWCNHGSLQPRPPGLLSDPSTSTFQGAGTTEPADRHPNRKARGQLRTKIESGEGTIPVRSNASIQTWDGVLQGERLLTMSCSDKIARWNVVGIQGSLLSIFVEPIYFSSIILGSLYHGDHLSRAMYQRISNIEDLPPLYTLNKPLLSGISNAEARQPGKAPNFSVNWTVGDSAIEVINATTGKDELGRASRLCKHALYCRWMRVHGKVPSHLLRSKITKPNVYHESKLAAKEYQAAKARLFTAFIKAGLGAWVEKPTEQDQFSLTP

SEQ ID NO: 4 - protein, 400 aa, artificial sequence
  the ADAR2-isoform2 effector domain
DQTPSRQPIPSEGLQLHLPQVLADAVSRLVLGKFGDLTDNFSSPHARRKVLAGVVMTTGTDVKDAKVISVSTGTKCINGEYMSDRGLALNDCHAEIISRRSLLRFLYTQLELYLNNKDDQKRSIFQKSERGGFRLKENVQFHLYISTSPCGDARIFSPHEPILEEPADRHPNRKARGQLRTKIESGEGTIPVRSNASIQTWDGVLQGERLLTMSCSDKIARWNVVGIQGSLLSIFVEPIYFSSIILGSLYHGDHLSRAMYQRISNIEDLPPLYTLNKPLLSGISNAEARQPGKAPNFSVNWTVGDSAIEVINATTGKDELGRASRLCKHALYCRWMRVHGKVPSHLLRSKITKPNVYHESKLAAKEYQAAKARLFTAFIKAGLGAWVEKPTEQDQFSLTP

SEQ ID NO: 5 - protein, 199 aa, artificial sequence
  APOBEC3A
MEASPASGPRHLMDPHIFTSNFNNGIGRHKTYLCYEVERLDNGTSVKMDQHRGFLHNQAKNLLCGFYGRHAELRFLDLVPSLQLDPAQIYRVTWFISWSPCFSWGCAGEVRAFLQENTHVRLRIFAARIYDYDPLYKEALQMLRDAGAQVSIMTYDEFKHCWDTFVDHQGCPFQPWDGLDEHSQALSGRLRAILQNQGN

SEQ ID NO: 6 - protein, 7 aa, artificial sequence
  the linker peptide
EFTGNGS

SEQ ID NO: 7 - protein, 16 aa, artificial sequence
  the linker peptide
SGSETPGTSESATPES

SEQ ID NO: 8 - protein, 20 aa, artificial sequence
  the linker peptide
DQTPSRQPIPSEGLQLHLPQ

SEQ ID NO: 9 - protein, 41 aa, artificial sequence
  the linker peptide
KAERMGFTEVTPVTGASLRRTMLLLSRSPEAQPKTLPLTGS

SEQ ID NO: 10 - protein, 1226 aa, artificial sequence
  ADAR1
MNPRQGYSLSGYYTHPFQGYEHRQLRYQQPGPGSSPSSFLLKQIEFLKGQLPEAPVIGKQTPSLPPSLPGLRPRFPVLLASSTRGRQVDIRGVPRGVHLRSQGLQRGFQHPSPRGRSLPQRGVDCLSSHFQELSIYQDQEQRILKFLEELGEGKATTAHDLSGKLGTPKKEINRVLYSLAKKGKLQKEAGTPPLWKIAVSTQAWNQHSGVVRPDGHSQGAPNSDPSLEPEDRNSTSVSEDLLEPFIAVSAQAWNQHSGVVRPDSHSQGSPNSDPGLEPEDSNSTSALEDPLEFLDMAEIKEKICDYLFNVSDSSALNLAKNIGLTKARDINAVLIDMERQGDVYRQGTTPPIWHLTDKKRERMQIKRNTNSVPETAPAAIPETKRNAEFLTCNIPTSNASNNMVTTEKVENGQEPVIKLENRQEARPEPARLKPPVHYNGPSKAGYVDFENGQWATDDIPDDLNSIRAAPGEFRAIMEMPSFYSHGLPRCSPYKKLTECQLKNPISGLLEYAQFASQTCEFNMIEQSGPPHEPRFKFQVVINGREFPPAEAGSKKVAKQDAAMKAMTILLEEAKAKDSGKSEESSHYSTEKESEKTAESQTPTPSATSFFSGKSPVTTLLECMHKLGNSCEFRLLSKEGPAHEPKFQYCVAVGAQTFPSVSAPSKKVAKQMAAEEAMKALHGEATNSMASDNQPEGMISESLDNLESMMPNKVRKIGELVRYLNTNPVGGLLEYARSHGFAAEFKLVDQSGPPHEPKFVYQAKVGGRWFPAVCAHSKKQGKQEAADAALRVLIGENEKAERMGFTEVTPVTGASLRRTMLLLSRSPEAQPKTLPLTGSTFHDQIAMLSHRCFNTLTNSFQPSLLGRKILAAIIMKKDSEDMGVVVSLGTGNRCVKGDSLSLKGETVNDCHAEIISRRGFIRFLYSELMKYNSQTAKDSIFEPAKGGEKLQIKKTVSFHLYISTAPCGDGALFDKSCSDRAMESTESRHYPVFENPKQGKLRTKVENGEGTIPVESSDIVPTWDGIRLGERLRTMSCSDKILRWNVLGLQGALLTHFLQPIYLKSVTLGYLFSQGHLTRAICCRVTRDGSAFEDGLRHPFIVNHPKVGRVSIYDSKRQSGKTKETSVNWCLADGYDLEILDGTRGTVDGPRNELSRVSKKNIFLLFKKLCSFRYRRDLLRLSYGEAKKAARDYETAKNYFKKGLKDMGYGNWISKPQEEKNFYLCPV

SEQ ID NO: 11 - protein, 741 aa, artificial sequence
  ADAR2-isoform1
MDIEDEENMSSSSTDVKENRNLDNVSPKDGSTPGPGEGSQLSNGGGGGPGRKRPLEEGSNGHSKYRLKKRRKTPGPVLPKNALMQLNEIKPGLQYTLLSQTGPVHAPLFVMSVEVNGQVFEGSGPTKKKAKLHAAEKALRSFVQFPNASEAHLAMGRTLSVNTDFTSDQADFPDTLFNGFETPDKAEPPFYVGSNGDDSFSSSGDLSLSASPVPASLAQPPLPVLPPFPPPSGKNPVMILNELRPGLKYDFLSESGESHAKSFVMSVVVDGQFFEGSGRNKKLAKARAAQSALAAIFNLHLDQTPSRQPIPSEGLQLHLPQVLADAVSRLVLGKFGDLTDNFSSPHARRKVLAGVVMTTGTDVKDAKVISVSTGTKCINGEYMSDRGLALNDCHAEIISRRSLLRFLYTQLELYLNNKDDQKRSIFQKSERGGFRLKENVQFHLYISTSPCGDARIFSPHEPILEGSRSYTQAGVQWCNHGSLQPRPPGLLSDPSTSTFQGAGTTEPADRHPNRKARGQLRTKIESGEGTIPVRSNASIQTWDGVLQGERLLTMSCSDKIARWNVVGIQGSLLSIFVEPIYFSSIILGSLYHGDHLSRAMYQRISNIEDLPPLYTLNKPLLSGISNAEARQPGKAPNFSVNWTVGDSAIEVINATTGKDELGRASRLCKHALYCRWMRVHGKVPSHLLRSKITKPNVYHESKLAAKEYQAAKARLFTAFIKAGLGAWVEKPTEQDQFSLTP

SEQ ID NO: 12 - protein, 701 aa, artificial sequence
  ADAR2-isoform2
MDIEDEENMSSSSTDVKENRNLDNVSPKDGSTPGPGEGSQLSNGGGGGPGRKRPLEEGSNGHSKYRLKKRRKTPGPVLPKNALMQLNEIKPGLQYTLLSQTGPVHAPLFVMSVEVNGQVFEGSGPTKKKAKLHAAEKALRSFVQFPNASEAHLAMGRTLSVNTDFTSDQADFPDTLFNGFETPDKAEPPFYVGSNGDDSFSSSGDLSLSASPVPASLAQPPLPVLPPFPPPSGKNPVMILNELRPGLKYDFLSESGESHAKSFVMSVVVDGQFFEGSGRNKKLAKARAAQSALAAIFNLHLDQTPSRQPIPSEGLQLHLPQVLADAVSRLVLGKFGDLTDNFSSPHARRKVLAGVVMTTGTDVKDAKVISVSTGTKCINGEYMSDRGLALNDCHAEIISRRSLLRFLYTQLELYLNNKDDQKRSIFQKSERGGFRLKENVQFHLYISTSPCGDARIFSPHEPILEEPADRHPNRKARGQLRTKIESGEGTIPVRSNASIQTWDGVLQGERLLTMSCSDKIARWNVVGIQGSLLSIFVEPIYFSSIILGSLYHGDHLSRAMYQRISNIEDLPPLYTLNKPLLSGISNAEARQPGKAPNFSVNWTVGDSAIEVINATTGKDELGRASRLCKHALYCRWMRVHGKVPSHLLRSKITKPNVYHESKLAAKEYQAAKARLFTAFIKAGLGAWVEKPTEQDQFSLTP

SEQ ID NO: 13 - protein, 7 aa, artificial sequence
  nuclear localization signal sequence
PKKKRKV

SEQ ID NO: 14 - protein, 36 aa, artificial sequence
  mitochondrial localization signal sequence
MLFNLRILLNNAAFRNGHNFMVRNFRCGQPLQNKVQ

(19) United States
(12) Patent Application Publication
(10) Pub. No.: US 2022/0195416 A1
(43) Pub. Date: Jun. 23, 2022

(54) RNA SITE-DIRECTED EDITING USING ARTIFICIALLY CONSTRUCTED RNA EDITING ENZYMES AND RELATED USES

(71) Applicant: SHANGHAI INSTITUTE OF NUTRITION AND HEALTH, CHINESE ACADEMY OF SCIENCES, Shanghai (CN)

(72) Inventors: Zefeng WANG, Shanghai (CN); Wenjian HAN, Shanghai (CN)

(73) Assignee: SHANGHAI INSTITUTE OF NUTRITION AND HEALTH, CHINESE ACADEMY OF SCIENCES, Shanghai (CN)

(21) Appl. No.: 17/601,963

(22) PCT Filed: Mar. 30, 2020
(86) PCT No.: PCT/CN2020/082183

§ 371 Date: Oct. 11, 2021

(30) Foreign Application Priority Data
Apr. 8, 2019 (CN) 201910277423.0

(51) Int. Cl. C12N 15/10; C12N 9/78; C12N 15/85; C12N 15/62; A61K 38/50

(52) U.S. Cl. CPC C12N 15/102; C12N 9/78; C12Y 305/04; A61K 48/00; C12N 15/62; A61K 38/50; C12N 15/85

(57) ABSTRACT

Disclosed are RNA site-directed editing using artificially constructed RNA editing enzymes and related uses. Provided is the fusion of an RNA recognition domain for binding RNA and a functional effector domain to form a new functional protein. The new functional protein specifically targets target RNA by means of the recognition domain and performs RNA editing using the effector domain.

### TECHNICAL FIELD

The present invention belongs to the field of biology. Specifically, the present invention relates to the use of artificially constructed RNA editing enzymes for RNA-directed editing and related applications.

### BACKGROUND

DNA is the most important genetic material in organisms. At present, a large part of the known human diseases are caused by genetic mutations, and single-base mutations are the largest category. Therefore, the development of a method to change the sequence of a single base in the genome and repair the pathogenic single base mutations efficiently and accurately is of great significance for the research and treatment of genetic diseases.

The current gene therapy strategies for single-base mutation diseases are mainly to treat diseases by directly repairing or replacing mutant genes at the DNA or RNA level. The main methods are the base editing systems ABE and CBE for DNA based on CRISPR technology. These technologies can perform base editing to a certain extent, but there are still many shortcomings:

(1) The current CRISPR-mediated base editing system has insufficient precision and low efficiency for single-base editing. There is a common editing window, and additional mutations will be introduced when editing the target site.

(2) The CRISPR system is very large. The current gene transfer technology is difficult to package the entire CRISPR system in the same system for delivery at one time. Therefore, there is inefficient gene transfer during the treatment process and the editing efficiency is low.

(3) Limited by the size of the transgene, some genomic loci are difficult to transfer.

(4) Immune response and toxicity caused by the Cas protein as a bacterial protein.

(5) Mutations during gene insertion or unexpected events during integration.

It is especially important that the long-term safety concerns of genomic DNA editing have always been a big problem because the changes in genomic DNA will accompany the cell for a lifetime.

Therefore, those skilled in the art urgently need to develop a method that can effectively edit genes in order to perform effective gene therapy on single-base mutation diseases.

### SUMMARY OF THE INVENTION

The purpose of the present invention is to provide a method and application that can effectively edit genes.

In a first aspect of the present invention, it provides an RNA editing enzyme, comprising:

(a) a RNA recognition domain, the RNA recognition domain is used to recognize the RNA recognition sequence of the RNA sequence to be edited, and bind to the RNA recognition sequence;

(b) a utility domain, which is used for nucleotide editing of the RNA sequence to be edited;

wherein, the RNA recognition domain and the utility binding domain are operably linked.

In another preferred embodiment, the utility domain is selected from the group consisting of the deamination catalytic domain of ADAR family members, the deamination catalytic domain of APOBEC family members, RNA methylase, RNA demethylase, added uracil synthase, and a combination thereof.

In another preferred embodiment, the RNA recognition domain contains n recognition units, and each recognition unit is used to recognize an RNA base, wherein n is a positive integer of 5-30.

In another preferred example, n is 6-24, more preferably 8-20, for example, 6, 7, 8, 9, 10, 11, 12, 13, 14, 15, 16, 17, 18, 19, 20, 21, 22, 23, 24.

In another preferred example, each recognition unit is an α-helix repeated sequence, and three amino acids at a specific position on the repeated sequence are responsible for binding RNA bases.

In another preferred example, the n recognition units are connected in series to form an RNA base recognition region.

In another preferred example, the RNA recognition domain further includes an optional protection region located on both sides of the RNA base recognition region to protect the RNA base recognition region.

In another preferred example, the RNA recognition domain is derived from PUF protein.

In another preferred example, the RNA recognition domain does not include the domain upstream of the RNA base recognition region in the PUF protein (except the protection region), but may or may not include the domain downstream of the RNA base recognition region in the PUF protein (except the protection region).

In another preferred example, the protection region is a non-functional repeated sequence at both ends of the RNA base recognition region.

In another preferred example, the RNA editing enzyme further includes one or more elements selected from the group consisting of linker peptide, tag sequence, signal peptide sequence, location peptide sequence, and a combination thereof.

In another preferred embodiment, the location peptide sequence includes a nuclear localization signal sequence, a mitochondrial localization signal sequence, and a combination thereof.

In another preferred example, the one or more elements are operably linked to the RNA recognition domain and utility domain.

In another preferred embodiment, the one or more elements are independently located at the N-terminal and/or C-terminal and/or middle of the RNA editing enzyme.

In another preferred embodiment, the signal peptide is located at the N-terminus of the RNA editing enzyme.

In another preferred example, the linker peptide, tag sequence, and/or location peptide signal is each independently located between the recognition domain and the utility domain.

In another preferred embodiment, the RNA is single-stranded.

In another preferred example, the RNA editing enzyme includes an RNA recognition domain and a utility domain, as well as an optional linker peptide, tag sequence, signal peptide sequence and/or location peptide sequence.

In another preferred example, the structure of the RNA editing enzyme is shown in any one of the following Formula I to formula IV:

D-L2-A-L1-B (I);

D-L2-B-L1-A (II);

A-L1-B-L2-D (III);

B-L1-A-L2-D (IV);

wherein each “-” is independently a linker peptide or a peptide bond;

A is a RNA recognition domain;

B is a utility domain;

L1 and L2 is each independently none or a linker peptide;

D is none or a location peptide.

In another preferred embodiment, the location peptide is located at the N-terminal, C-terminal or middle of the RNA editing enzyme.

In another preferred embodiment, the RNA recognition domain is a domain capable of recognizing and binding RNA, preferably from an RNA binding protein.

In another preferred embodiment, the RNA recognition domain is a PUF protein fragment.

In another preferred embodiment, the PUF protein fragment is a fragment of the PUF protein that can recognize the RNA-binding domain.

In another preferred embodiment, the PUF protein includes a PUF protein and a homologous protein thereof.

In another preferred embodiment, the PUF protein is derived from a mammal, preferably from a primate, and more preferably from a human.

In another preferred embodiment, the RNA recognition domain has an amino acid sequence as shown in SEQ ID NO.: 1.

(SEQ ID NO.: 1)
GRSRLLEDFRNNRYPNLQLREIAGHIMEFSQDQHGSRFIQLKLERATPAE
RQLVFNEILQAAYQLMVDVFGNYVIQKFFEFGSLEQKLALAERIRGHVLS
LALQMYGCRVIQKALEFIPSDQQNEMVRELDGHVLKCVKDQNGNHVVQKC
IECVQPQSLQFIIDAFKGQVFALSTHPYGCRVIQRILEHCLPDQTLPILE
ELHQHTEQLVQDQYGNYVIQHVLEHGRPEDKSKIVAEIRGNVLVLSQHKF
ASNVVEKCVTHASRTERAVLIDEVCTMNDGPHSALYTMMKDQYANYVVQK
MIDVAEPGQRKIVMHKIRPHIATLRKYTYGKHILAKLEKYYMKNGVDLG

In another preferred embodiment, the ADAR family member includes: dADAR, ADAR1, ADAR2, TadA, and a combination thereof.

In another preferred embodiment, the member of the ADAR family is derived from a human or Drosophila or bacteria.

In another preferred embodiment, the ADAR family member includes: Drosophila ADAR, human ADAR1, human ADAR2, E. coli TadA, or a combination thereof.

In another preferred embodiment, the ADAR1 includes a natural ADAR1 and ADAR1 mutant.

In another preferred example, the ADAR1 mutant has a mutation at position 1008 in the amino acid sequence corresponding to the natural ADAR1; preferably, the glutamic acid (E) at position 1008 is mutated to glutamine (Q).

In another preferred example, the effector domain is derived from ADAR1 and has the amino acid sequence as shown in SEQ ID NO.: 2:

(SEQ ID NO.: 2)
KAERMGFTEVTPVTGASLRRTMLLLSRSPEAQPKTLPLTGSTFHDQIAML
SHRCFNTLTNSFQPSLLGRKILAAIIMKKDSEDMGVVVSLGTGNRCVKGD
SLSLKGETVNDCHAEIISRRGFIRFLYSELMKYNSQTAKDSIFEPAKGGE
KLQIKKTVSFHLYISTAPCGDGALFDKSCSDRAMESTESRHYPVFENPKQ
GKLRTKVENGEGTIPVESSDIVPTWDGIRLGERLRTMSCSDKILRWNVLG
LQGALLTHFLQPIYLKSVTLGYLFSQGHLTRAICCRVTRDGSAFEDGLRH
PFIVNHPKVGRVSIYDSKRQSGKTKETSVNWCLADGYDLEILDGTRGTVD
GPRNELSRVSKKNIFLLFKKLCSFRYRRDLLRLSYGEAKKAARDYETAKN
YFKKGLKDMGYGNWISKPQEEKNFYLCPV

or the effector domain is the ADAR1-E1008Q effector domain, and its amino acid sequence is the same as SEQ ID No.: 2, but the position 211 in SEQ ID No.: 2 is mutated from E to Q:

(SEQ ID No.: 2, among them, the position 211 is
mutated from E to Q)
KAERMGFTEVTPVTGASLRRTMLLLSRSPEAQPKTLPLTGSTFHDQIAML
SHRCFNTLTNSFQPSLLGRKILAAIIMKKDSEDMGVVVSLGTGNRCVKGD
SLSLKGETVNDCHAEIISRRGFIRFLYSELMKYNSQTAKDSIFEPAKGGE
KLQIKKTVSFHLYISTAPCGDGALFDKSCSDRAMESTESRHYPVFENPKQ
GKLRTKVENGQGTIPVESSDIVPTWDGIRLGERLRTMSCSDKILRWNVLG
LQGALLTHFLQPIYLKSVTLGYLFSQGHLTRAICCRVTRDGSAFEDGLRH
PFIVNHPKVGRVSIYDSKRQSGKTKETSVNWCLADGYDLEILDGTRGTVD
GPRNELSRVSKKNIFLLFKKLCSFRYRRDLLRLSYGEAKKAARDYETAKN
YFKKGLKDMGYGNWISKPQEEKNFYLCPV.

In another preferred embodiment, the ADAR2 mutant is mutated at position 488 in the amino acid sequence corresponding to the natural ADAR2; preferably, the glutamate (E) at position 488 is mutated to glutamine (Q).

In another preferred example, the effector domain has the amino acid sequence as shown in SEQ ID NO.: 2, 3, or 4 or its derivative sequence.

In another preferred example, the derivative sequence includes: SEQ ID No.: 2, and the position 211 is mutated from E to Q); SEQ ID No.: 3, wherein the position 227 is mutated from E to Q; SEQ ID No.: 4, wherein the position 187 is mutated from E to Q.

In another preferred embodiment, the effector domain is selected from the group consisting of:

(i) an ADAR2-isoform1 effector domain with the amino acid sequence as shown in SEQ ID NO.: 3

(ii) an ADAR2-isoform1-E488Q effector domain having the amino acid sequence as shown in SEQ ID NO.: 3 and mutated from E to Q at position 227; and

(iii) an ADAR2-isoform2 effector domain having the amino acid sequence as shown in SEQ ID NO.: 4;

(iv) an ADAR2-isoform2-E488Q effector domain having the amino acid sequence as shown in SEQ ID NO.: 4 and mutated from E to Q at position 187;

In another preferred example, the amino acid sequence of the effector domain of ADAR2-isoform1 is shown as follows:

(SEQ ID NO.: 3)
DQTPSRQPIPSEGLQLHLPQVLADAVSRLVLGKFGDLTDNFSSPHARRKV
LAGVVMTTGTDVKDAKVISVSTGTKCINGEYMSDRGLALNDCHAEIISRR
SLLRFLYTQLELYLNNKDDQKRSIFQKSERGGFRLKENVQFHLYISTSPC
GDARIFSPHEPILEGSRSYTQAGVQWCNHGSLQPRPPGLLSDPSTSTFQG
AGTTEPADRHPNRKARGQLRTKIESGEGTIPVRSNASIQTWDGVLQGERL
LTMSCSDKIARWNVVGIQGSLLSIFVEPIYFSSIILGSLYHGDHLSRAMY
QRISNIEDLPPLYTLNKPLLSGISNAEARQPGKAPNFSVNWTVGDSAIEV
INATTGKDELGRASRLCKHALYCRWMRVHGKVPSHLLRSKITKPNVYHES
KLAAKEYQAAKARLFTAFIKAGLGAWVEKPTEQDQFSLTP

In another preferred example, the amino acid sequence of the ADAR2-isoform1-F4220 effector domain is shown as follows:

DQTPSRQPIPSEGLQLHLPQVLADAVSRLVLGKFGDLTDNFSSPHARRKV
LAGVVMTTGTDVKDAKVISVSTGTKCINGEYMSDRGLALNDCHAEIISRR
SLLRFLYTQLELYLNNKDDQKRSIFQKSERGGFRLKENVQFHLYISTSPC
GDARIFSPHEPILEGSRSYTQAGVQWCNHGSLQPRPPGLLSDPSTSTFQG
AGTTEPADRHPNRKARGQLRTKIESGQGTIPVRSNASIQTWDGVLQGERL
LTMSCSDKIARWNVVGIQGSLLSIFVEPIYFSSIILGSLYHGDHLSRAMY
QRISNIEDLPPLYTLNKPLLSGISNAEARQPGKAPNFSVNWTVGDSAIEV
INATTGKDELGRASRLCKHALYCRWMRVHGKVPSHLLRSKITKPNVYHES
KLAAKEYQAAKARLFTAFIKAGLGAWVEKPTEQDQFSLTP

(the yellow amino acid is the position 488 amino acid mutation site) (SEQ ID No.: 3, wherein the position 227 is mutated from E to Q)

In another preferred example, the amino acid sequence of the ADAR2-isoform2 effector domain is as follows:

(SEQ ID NO.: 4)
DQTPSRQPIPSEGLQLHLPQVLADAVSRLVLGKFGDLTDNFSSPHARRKV
LAGVVMTTGTDVKDAKVISVSTGTKCINGEYMSDRGLALNDCHAEIISRR
SLLRFLYTQLELYLNNKDDQKRSIFQKSERGGFRLKENVQFHLYISTSPC
GDARIFSPHEPILEEPADRHPNRKARGQLRTKIESGEGTIPVRSNASIQT
WDGVLQGERLLTMSCSDKIARWNVVGIQGSLLSIFVEPIYFSSIILGSLY
HGDHLSRAMYQRISNIEDLPPLYTLNKPLLSGISNAEARQPGKAPNFSVN
WTVGDSAIEVINATTGKDELGRASRLCKHALYCRWMRVHGKVPSHLLRSK
ITKPNVYHESKLAAKEYQAAKARLFTAFIKAGLGAWVEKPTEQDQFSLTP

In another preferred example, the amino acid sequence of the ADAR2-isoform2-E488Q effector domain is as follows:

DQTPSRQPIPSEGLQLHLPQVLADAVSRLVLGKFGDLTDNFSSPHARRKV
LAGVVMTTGTDVKDAKVISVSTGTKCINGEYMSDRGLALNDCHAEIISRR
SLLRFLYTQLELYLNNKDDQKRSIFQKSERGGFRLKENVQFHLYISTSPC
GDARIFSPHEPILEEPADRHPNRKARGQLRTKIESGQGTIPVRSNASIQT
WDGVLQGERLLTMSCSDKIARWNVVGIQGSLLSIFVEPIYFSSIILGSLY
HGDHLSRAMYQRISNIEDLPPLYTLNKPLLSGISNAEARQPGKAPNFSVN
WTVGDSAIEVINATTGKDELGRASRLCKHALYCRWMRVHGKVPSHLLRSK
ITKPNVYHESKLAAKEYQAAKARLFTAFIKAGLGAWVEKPTEQDQFSLTP

(yellow amino acid is the position 488 amino acid mutation site) (SEQ ID No.: 4, the position 187 is mutated from E to Q)

In another preferred embodiment, the APOBEC family member includes: Apobec1, Apobec3A, Apobec3G, or a combination thereof.

In another preferred embodiment, the APOBEC family member is derived from a human or mouse (rat), preferably from a human.

In another preferred embodiment, the APOBEC family member is selected from the group consisting of human Apobec1, human Apobec3A, human Apobec3B, human Apobec3C, human Apobec3D, human Apobec3F, human Apobec3G, human Apobec3H, human AID, mouse Apobec1, mouse Apobec3A, mouse AID, rat Apobec1, rat Apobec3A, rat AID, and a combination thereof.

In another preferred example, the APOBEC3A has the amino acid sequence as shown in SEQ ID NO.: 5 (full-length amino acid sequence).

(SEQ ID NO.: 5)
MEASPASGPRHLMDPHIFTSNFNNGIGRHKTYLCYEVERLDNGTSVKMDQ
HRGFLHNQAKNLLCGFYGRHAELRFLDLVPSLQLDPAQIYRVTWFISWSP
CFSWGCAGEVRAFLQENTHVRLRIFAARIYDYDPLYKEALQMLRDAGAQV
SIMTYDEFKHCWDTFVDHQGCPFQPWDGLDEHSQALSGRLRAILQNQGN.

In another preferred embodiment, the RNA methylase includes: METTL3, METTL14, and a combination thereof.

In another preferred embodiment, the RNA demethylase is Alpha-ketoglutarate-dependent dioxygenase FTO.

In another preferred embodiment, the added uracil synthase is pseudoouridine7.

In another preferred example, the editing window of the RNA editing enzyme is position 7-14, preferably position 8-13, and more preferably position 9-11, wherein the calculation starts from the first position of the 5′ end of the PUF binding site (that is, the first position).

In another preferred embodiment, the RNA editing enzyme does not contain RNA and/or DNA.

In another preferred embodiment, the RNA recognition domain and the utility domain in the RNA editing enzyme are connected in a head-to-tail, head-to-head, tail-to-head, or tail-to-tail manner.

In another preferred embodiment, the RNA recognition domain and the utility domain in the RNA editing enzyme are connected directly or through a linker peptide.

In another preferred example, the C-terminal or N-terminal of the RNA editing enzyme further includes an NLS sequence and an MLS sequence.

NLS nuclear localization signal sequence: PKKKRKV (SEQ ID No.: 13).

MLS mitochondrial localization signal sequence:

(SEQ ID No.: 14)
MLFNLRILLNNAAFRNGHNFMVRNFRCGQPLQNKVQ.

In another preferred embodiment, the linker peptide is a none or a flexible peptide.

In another preferred embodiment, the linker peptide is selected from the group consisting of Linker2, Linker7, XTEN, Linker20, Linker40, and a combination thereof.

In another preferred example, the length of the linker peptide is 0-40 aa, preferably 2-20 aa.

In another preferred example, the linker peptide has an amino acid sequence as shown in any one of SEQ ID NO.: 6-9:

Linker2: EF
Linker7:
(SEQ ID NO.: 6)
EFTGNGS
XTEN:
(SEQ ID NO.: 7)
SGSETPGTSESATPES
Linker20:
(SEQ ID NO.: 8)
DQTPSRQPIPSEGLQLHLPQ
Linker41:
(SEQ ID NO.: 9)
KAERMGFTEVTPVTGASLRRTMLLLSRSPEAQPKTLPLTGS

In a second aspect of the present invention, it provides an isolated polynucleotide which encodes the RNA editing enzyme according to the first aspect of the present invention.

In a third aspect of the present invention, it provides a vector, which comprises the polynucleotide according to the second aspect of the present invention.

In another preferred embodiment, the vector includes DNA and RNA.

In another preferred embodiment, the vector is selected from the group consisting of: plasmid, viral vector, transposon, and a combination thereof.

In another preferred embodiment, the vector includes DNA virus and retroviral vector.

In another preferred embodiment, the vector is selected from the group consisting of a lentiviral vector, an adenovirus vector, an adeno-associated virus vector, and a combination thereof.

In another preferred embodiment, the vector is a lentiviral vector.

In another preferred embodiment, the vector includes one or more promoters, which are operably linked to the nucleic acid sequence, enhancer, intron, transcription termination signal, polyadenylation sequence, origin of replication, selected marker, nucleic acid restriction site, and/or homologous recombination site.

In another preferred example, the vector is a vector containing or inserted with the polynucleotide of the second aspect of the present invention.

In a fourth aspect of the present invention, it provides a host cell, which contains the vector according to the third aspect of the present invention, or the exogenous polynucleotide according to the second aspect of the present invention integrated into the chromosome, or express the RNA editing enzyme according to the first aspect of the present invention.

In another preferred embodiment, the host cell is a prokaryotic cell or a eukaryotic cell.

In another preferred embodiment, the host cell is a human cell or a non-human mammalian cell.

In a fifth aspect of the present invention, it provides a preparation, wherein the preparation comprises the RNA editing enzyme according to the first aspect of the present invention, or the polynucleotide according to the second aspect, or the vector according to the third aspect, and a pharmaceutically acceptable carrier or excipient.

In another preferred embodiment, the preparation is a liquid preparation.

In a sixth aspect of the present invention, it provides a use of the RNA editing enzyme according to the first aspect, or the polynucleotide according to the second aspect, or the host cell according to the fourth aspect of the present invention for the preparation of

(a) a drug or preparation for gene therapy; and/or

(b) a reagent for editing RNA.

In another preferred example, the editing RNA includes mutating A to G and/or mutating C to U in RNA.

In a seventh aspect of the present invention, it provides a method for editing RNA, the method comprising the steps:

(1) providing RNA to be edited and the RNA editing enzyme according to the first aspect of the present invention; and

(2) using the RNA editing enzyme of claim 1 to edit the RNA.

In another preferred embodiment, the method is in vitro or in vivo.

In another preferred embodiment, the method is for non-diagnostic and non-therapeutic purposes.

In another preferred example, the editing RNA includes mutating A to G and/or mutating C to U in RNA.

In a eighth aspect of the present invention, it provides a method for preparing the RNA editing enzyme of the first aspect of the present invention, the method comprising the steps:

under a suitable expression condition, culturing the host cell according to the fourth aspect of the present invention, thereby expressing the RNA editing enzyme; and

isolating the RNA editing enzyme.

In another preferred embodiment, the host cell is a prokaryotic cell or a eukaryotic cell.

In a ninth aspect of the present invention, it provides a method for treating diseases, the method comprising: administering the RNA editing enzyme according to the first aspect of the present invention or the polynucleotide according to the second aspect of the present invention or the vector according to the third aspect of the present invention, or the preparation of the fifth aspect of the present invention to a subject in need.

It should be understood that, within the scope of the present invention, each technical feature of the present invention described above and in the following (as examples) may be combined with each other to form a new or preferred technical solution, which is not listed here due to space limitations.

### DESCRIPTION OF FIGURE

FIG. 1 shows the construction of different A→G RNA editing enzymes.

FIG. 2 shows the use of PARSEs to edit target RNA.

FIG. 3 shows that the enzyme produced by replacing the positions of PUF and ADAR cannot edit the target RNA.

FIG. 4 shows the efficiency and off-target rate analysis of PARSEs on target RNA editing.

FIG. 5 shows the use of ePARSE1 to repair the abnormal RNA editing event of the GRIA2 gene.

FIG. 6 shows the use of ePARSE2 to repair disease-causing point mutations in related genes.

FIG. 7 shows that extending the PUF recognition domain in the PARSE system can significantly improve the RNA editing accuracy of the PARSE system.

FIG. 8 shows the construction of APRSEs to edit target RNA.

FIG. 9 shows the use of APRSE to repair disease-causing point mutations in related genes.

FIG. 10 shows that extending the PUF recognition domain in the APRSE system can significantly improve the RNA editing accuracy of the APRSE system.

FIG. 11 shows a schematic diagram of the structure of the PUF element.

### DETAILED DESCRIPTION

After extensive and in-depth research, the inventors have developed an RNA editing enzyme with a unique structure for the first time. The inventors have unexpectedly discovered that a novel RNA editing enzyme based on the RNA-binding recognition domain and utility domain can very effectively target specific RNA regions and perform efficient and accurate RNA editing. The RNA editing enzyme of the present invention can not only perform RNA editing efficiently and accurately, but also can effectively prevent back mutation, so that it has the advantages of more flexibility, safety, efficiency and the like. On this basis, the inventors have completed the present invention.

### Term Description

Unless otherwise defined, all technical and scientific terms used herein have the same meaning as commonly understood by those of ordinary skill in the art to which the present invention belongs.

As used herein, when used in reference to a specifically recited value, the term “about” means that the value can vary from the recited value by no more than 1%. For example, as used herein, the expression “about 100” includes all values between 99 and 101 (e.g., 99.1, 99.2, 99.3, 99.4, etc.).

As used herein, the term “containing” or “comprising (including)” can be open, semi-closed, and closed. In other words, the term also includes “substantially consisting of” or “consisting of”.

PUF Protein (Pumilio Homolog 1)

PUF protein is a sequence-specific RNA binding protein with a conserved RNA binding domain, which regulates the stability or translation efficiency of mRNA by binding to the 3′-UTR of the target mRNA. A typical PUF binding domain contains 8 α-helix repeated sequence, each repeated sequence is 36 amino acids, responsible for identifying and binding 1 base, at both ends of the binding domain, each has a non-functional repeated sequence to protect the 8 repeated sequences in the middle. Three amino acids at specific positions on the repeated sequence of α-helix are responsible for binding RNA bases and wherein the side chain amino acids at positions 12 and 16 bind to RNA bases through hydrogen bonds, and the amino acid at position 13 serves as an auxiliary binding. Different combinations of amino acids are responsible for recognizing different bases (see FIG. 11). After modification and design of each repeated sequence of the PUF protein, the newly designed PUF of this application can recognize and bind any 8-base RNA sequence. After modification, the number of PUF repeated sequences can be increased and decreased to expand the PUF's ability to bind RNA, so that the PUF of the present application can recognize 6 to 16 bases, and further can recognize 20 bases. In different species, PUF protein has many homologous proteins, which have similar sequence characteristics and similar functions to PUF protein.

In a preferred example of the present invention, human-derived PUM1 is selected as a tool for RNA recognition.

ADAR

ADAR (Double-stranded RNA-specific adenosine deaminase) protein is a type of RNA deaminase that acts on double-stranded RNA. It catalyzes the hydrolysis and deamination of adenosine in double-stranded RNA to form inosine (adenosine-to-inosine, A-to-I), known as A-to-I RNA editing, inosine (I) is recognized as guanosine (G) during translation, realizing A-to-G RNA editing. The main members of the protein family are ADAR1, ADAR2 and ADAR3. The gene editing produced by this enzyme will affect the expression and function of genes, including by changing the mRNA translation of codons, thereby changing the amino acid sequence of the protein; by changing the splice site recognition sequence, thereby performing pre-mRNA splicing; achieving RNA stability by changing the sequence involved in nuclease recognition; RNA viral genome changes sequence during viral RNA replication to achieve genetic stability and regulate some structure-dependent RNA metabolic activities, such as microRNA production, targeting or protein-RNA interaction.

Representative ADARs include ADAR1, ADAR2-isoform1 and ADAR2-isoform1 or the homologous proteins thereof.

A representative full-length amino acid sequence of ADAR1 is as follows:

(SEQ ID No.: 10)
MNPRQGYSLSGYYTHPFQGYEHRQLRYQQPGPGSSPSSFLLKQIEFLKGQ
LPEAPVIGKQTPSLPPSLPGLRPRFPVLLASSTRGRQVDIRGVPRGVHLR
SQGLQRGFQHPSPRGRSLPQRGVDCLSSHFQELSIYQDQEQRILKFLEEL
GEGKATTAHDLSGKLGTPKKEINRVLYSLAKKGKLQKEAGTPPLWKIAVS
TQAWNQHSGVVRPDGHSQGAPNSDPSLEPEDRNSTSVSEDLLEPFIAVSA
QAWNQHSGVVRPDSHSQGSPNSDPGLEPEDSNSTSALEDPLEFLDMAEIK
EKICDYLFNVSDSSALNLAKNIGLTKARDINAVLIDMERQGDVYRQGTTP
PIWHLTDKKRERMQIKRNTNSVPETAPAAIPETKRNAEFLTCNIPTSNAS
NNMVTTEKVENGQEPVIKLENRQEARPEPARLKPPVHYNGPSKAGYVDFE
NGQWATDDIPDDLNSIRAAPGEFRAIMEMPSFYSHGLPRCSPYKKLTECQ
LKNPISGLLEYAQFASQTCEFNMIEQSGPPHEPRFKFQVVINGREFPPAE
AGSKKVAKQDAAMKAMTILLEEAKAKDSGKSEESSHYSTEKESEKTAESQ
TPTPSATSFFSGKSPVTTLLECMHKLGNSCEFRLLSKEGPAHEPKFQYCV
AVGAQTFPSVSAPSKKVAKQMAAEEAMKALHGEATNSMASDNQPEGMISE
SLDNLESMMPNKVRKIGELVRYLNTNPVGGLLEYARSHGFAAEFKLVDQS
GPPHEPKFVYQAKVGGRWFPAVCAHSKKQGKQEAADAALRVLIGENEKAE
RMGFTEVTPVTGASLRRTMLLLSRSPEAQPKTLPLTGSTFHDQIAMLSHR
CFNTLTNSFQPSLLGRKILAAIIMKKDSEDMGVVVSLGTGNRCVKGDSLS
LKGETVNDCHAEIISRRGFIRFLYSELMKYNSQTAKDSIFEPAKGGEKLQ
IKKTVSFHLYISTAPCGDGALFDKSCSDRAMESTESRHYPVFENPKQGKL
RTKVENGEGTIPVESSDIVPTWDGIRLGERLRTMSCSDKILRWNVLGLQG
ALLTHFLQPIYLKSVTLGYLFSQGHLTRAICCRVTRDGSAFEDGLRHPFI
VNHPKVGRVSIYDSKRQSGKTKETSVNWCLADGYDLEILDGTRGTVDGPR
NELSRVSKKNIFLLFKKLCSFRYRRDLLRLSYGEAKKAARDYETAKNYFK
KGLKDMGYGNWISKPQEEKNFYLCPV*

A representative full-length amino acid sequence of ADAR2-isoform1 is as follows:

(SEQ ID No.: 11)
MDIEDEENMSSSSTDVKENRNLDNVSPKDGSTPGPGEGSQLSNGGGGGPG
RKRPLEEGSNGHSKYRLKKRRKTPGPVLPKNALMQLNEIKPGLQYTLLSQ
TGPVHAPLFVMSVEVNGQVFEGSGPTKKKAKLHAAEKALRSFVQFPNASE
AHLAMGRTLSVNTDFTSDQADFPDTLFNGFETPDKAEPPFYVGSNGDDSF
SSSGDLSLSASPVPASLAQPPLPVLPPFPPPSGKNPVMILNELRPGLKYD
FLSESGESHAKSFVMSVVVDGQFFEGSGRNKKLAKARAAQSALAAIFNLH
LDQTPSRQPIPSEGLQLHLPQVLADAVSRLVLGKFGDLTDNFSSPHARRK
VLAGVVMTTGTDVKDAKVISVSTGTKCINGEYMSDRGLALNDCHAEIISR
RSLLRFLYTQLELYLNNKDDQKRSIFQKSERGGFRLKENVQFHLYISTSP
CGDARIFSPHEPILEGSRSYTQAGVQWCNHGSLQPRPPGLLSDPSTSTFQ
GAGTTEPADRHPNRKARGQLRTKIESGEGTIPVRSNASIQTWDGVLQGER
LLTMSCSDKIARWNVVGIQGSLLSIFVEPIYFSSIILGSLYHGDHLSRAM
YQRISNIEDLPPLYTLNKPLLSGISNAEARQPGKAPNFSVNWTVGDSAIE
VINATTGKDELGRASRLCKHALYCRWMRVHGKVPSHLLRSKITKPNVYHE
SKLAAKEYQAAKARLFTAFIKAGLGAWVEKPTEQDQFSLTP*

A representative full-length amino acid sequence of ADAR2-isoform2 is as follows:

(SEQ ID No.: 12)
MDIEDEENMSSSSTDVKENRNLDNVSPKDGSTPGPGEGSQLSNGGGGGPG
RKRPLEEGSNGHSKYRLKKRRKTPGPVLPKNALMQLNEIKPGLQYTLLSQ
TGPVHAPLFVMSVEVNGQVFEGSGPTKKKAKLHAAEKALRSFVQFPNASE
AHLAMGRTLSVNTDFTSDQADFPDTLFNGFETPDKAEPPFYVGSNGDDSF
SSSGDLSLSASPVPASLAQPPLPVLPPFPPPSGKNPVMILNELRPGLKYD
FLSESGESHAKSFVMSVVVDGQFFEGSGRNKKLAKARAAQSALAAIFNLH
LDQTPSRQPIPSEGLQLHLPQVLADAVSRLVLGKFGDLTDNFSSPHARRK
VLAGVVMTTGTDVKDAKVISVSTGTKCINGEYMSDRGLALNDCHAEIISR
RSLLRFLYTQLELYLNNKDDQKRSIFQKSERGGFRLKENVQFHLYISTSP
CGDARIFSPHEPILEEPADRHPNRKARGQLRTKIESGEGTIPVRSNASIQ
TWDGVLQGERLLTMSCSDKIARWNVVGIQGSLLSIFVEPIYFSSIILGSL
YHGDHLSRAMYQRISNIEDLPPLYTLNKPLLSGISNAEARQPGKAPNFSV
NWTVGDSAIEVINATTGKDELGRASRLCKHALYCRWMRVHGKVPSHLLRS
KITKPNVYHESKLAAKEYQAAKARLFTAFIKAGLGAWVEKPTEQDQFSLT
P*

APOBECs Protein

APOBEC (apolipoprotein B mRNA editing enzyme, catalytic polypeptide-like) is an evolutionary conserved cytidine deaminase, which acts on the single-stranded region of DNA/RNA and catalyzes the hydrolysis and deamination of cytidine on single-stranded DNA/RNA to form uridine (cytosine-to-uracil, C-to-U), realizing C-to-U DNA/RNA editing. The main members of this protein family are AID, Apobec1, Apobec2, Apobec3A, Apobec3B, Apobec3C, Apobec3D, Apobec3F, Apobec3G, Apobec3H and Apobec4.

RNA Editing Enzyme

As used herein, “RNA editing enzyme of the present invention”, “artificial RNA editing enzyme”, “fusion protein” or “polypeptide of the present invention” all refer to the RNA editing enzyme described in the first aspect of the present invention.

The RNA editing enzyme of the present invention includes:

(a) a RNA recognition domain, the RNA recognition domain is used to recognize the RNA recognition sequence of the RNA sequence to be edited, and bind to the RNA recognition sequence;

(b) a utility domain, which is used for nucleotide editing of the RNA sequence to be edited;

wherein, the RNA recognition domain and the utility binding domain are operably connected.

As used herein, “operable (operably) connected (to)” or “operable (operably) linked (to)” refers to a parallel relationship in which the elements are in a relationship that allows them to function as expected. For example, if the RNA recognition domain and the utility domain are connected (directly connected, or connected through a connecting element, or connected through other functional elements located between the two), then as long as the RNA recognition domain and utility domain can perform their respective functions, that is, the RNA recognition domain recognizes and binds to a predetermined RNA recognition sequence, and the utility domain can perform nucleotide editing on the RNA sequence to be edited, and the two are operatively connected. Similarly, if a promoter can cause transcription or expression of a coding sequence, the promoter is operably linked to the coding sequence.

As used herein, the term “RNA editing enzyme of the present invention” also includes variant forms of the sequence having the above-mentioned activity. These variant forms include (but are not limited to): 1-3 (usually 1-2, more preferably 1) amino acid deletions, insertions and/or substitutions, and adding or deleting one or several (usually within 3, preferably within 2, more preferably within 1) amino acid at the C-terminal and/or N-terminal. For example, in this field, substitution with close or similar amino acids usually does not change the function of the protein. For another example, adding or deleting one or several amino acids at the C-terminus and/or N-terminus usually does not change the structure and function of the protein. In addition, the term also includes the polypeptide of the present invention in monomeric and multimeric forms. The term also includes linear and non-linear polypeptides (such as cyclic peptides).

The present invention also includes active fragments, derivatives and analogs of the above RNA editing enzymes. As used herein, the terms “fragment”, “derivative” and “analog” refer to a polypeptide that substantially retains the function or activity of the RNA editing enzyme of the present invention. The polypeptide fragments, derivatives or analogues of the present invention can be (i) a polypeptide with one or more conservative or non-conservative amino acid residues (preferably conservative amino acid residues) substituted, or (ii) a polypeptide with substitution groups in one or more amino acid residues, or (iii) a polypeptide formed by fusion of an antigenic peptide with another compound (such as a compound that extends the half-life of the viral capsid protein mutant, such as polyethylene glycol), or (iv) an additional amino acid sequence is fused to this polypeptide sequence to form a polypeptide (fusion protein formed by fusion with leader sequence, secretory sequence or 6His and other tag sequences). According to the teachings herein, these fragments, derivatives and analogs fall within the scope of those skilled in the art.

A preferred type of active derivative means that compared with the amino acid sequence of Formula I, there are at most 3, preferably at most 2, and more preferably at most 1 amino acid replaced by an amino acid with close or similar properties to form a polypeptide. These conservative variant polypeptides are best produced according to Table A by performing amino acid substitutions.

TABLE A

| | | Preferred |
|---|---|---|
| Initial residues | Representative substitution | substitution |
| Ala (A) | Val; Leu; Ile | Val |
| Arg (R) | Lys; Gln; Asn | Lys |
| Asn (N) | Gln; His; Lys; Arg | Gln |
| Asp (D) | Glu | Glu |
| Cys (C) | Ser | Ser |
| Gln (Q) | Asn | Asn |
| Glu (E) | Asp | Asp |
| Gly (G) | Pro; Ala | Ala |
| His (H) | Asn; Gln; Lys; Arg | Arg |
| Ile (I) | Leu; Val; Met; Ala; Phe | Leu |
| Leu (L) | Ile; Val; Met; Ala; Phe | Ile |
| Lys (K) | Arg; Gln; Asn | Arg |
| Met (M) | Leu; Phe; Ile | Leu |
| Phe (F) | Leu; Val; Ile; Ala; Tyr | Leu |
| Pro (P) | Ala | Ala |
| Ser (S) | Thr | Thr |
| Thr (T) | Ser | Ser |
| Trp (W) | Tyr; Phe | Tyr |
| Tyr (Y) | Trp; Phe; Thr; Ser | Phe |
| Val (V) | Ile; Leu; Met; Phe; Ala | Leu |

The present invention also provides analogs of the RNA editing enzyme of the present invention. The difference between these analogs and the polypeptide as shown in SEQ ID NO.: 8, 9 or 13 may be a difference in amino acid sequence, or a difference in modified form that does not affect the sequence, or both. Analogs also include analogs having residues different from natural L-amino acids (such as D-amino acids), and analogs having non-naturally occurring or synthetic amino acids (such as β, γ-amino acids). It should be understood that the polypeptide of the present invention is not limited to the representative polypeptides as exemplified above.

Modified (usually unchanged primary structure) forms include: chemically derived forms of peptides in vivo or in vitro, such as acetylation or carboxylation. Modifications also include glycosylation, such as those produced by glycosylation modification during the synthesis and processing or during further processing steps of peptides. This modification can be accomplished by exposing the peptides to an enzyme that performs glycosylation (such as mammalian glycosylase or deglycosylase). Modified forms also include sequences with phosphorylated amino acid residues (such as phosphotyrosine, phosphoserine, phosphothreonine). It also includes peptides that have been modified to improve their resistance to proteolysis or to optimize their solubility.

Coding Sequence

The present invention also relates to polynucleotides encoding RNA editing enzymes of the present invention.

The polynucleotide of the present invention may be in the form of DNA or RNA. DNA can be a coding strand or a non-coding strand. The full-length nucleotide sequence of the present invention or its fragments can usually be obtained by PCR amplification method, recombination method or artificial synthesis method. At present, the DNA sequence encoding the polypeptide (or fragment or derivative thereof) of the present invention can be obtained completely through chemical synthesis. The DNA sequence can then be introduced into various existing DNA molecules (or such as vectors) and cells known in the art.

The present invention also relates to a vector containing the polynucleotide of the present invention, and a host cell produced by genetic engineering using the vector or polypeptide coding sequence of the present invention. The aforementioned polynucleotides, vectors or host cells may be isolated.

As used herein, “isolated” refers to the separation of a substance from its original environment (if it is a natural substance, the original environment is the natural environment). For example, the polynucleotides and polypeptides in the natural state in living cells are not separated and purified, but the same polynucleotides or polypeptides are separated and purified if they are separated from other substances that co-exist in the natural state.

The polynucleotide of the present invention may be in the form of DNA or RNA. The form of DNA includes cDNA, genomic DNA or synthetic DNA. DNA can be single-stranded or double-stranded. DNA can be a coding strand or a non-coding strand.

The present invention also relates to variants of the above-mentioned polynucleotides, which encode protein fragments, analogs and derivatives having the same amino acid sequence as the present invention. The variants of this polynucleotide can be naturally occurring allelic variants or non-naturally occurring variants. These nucleotide variants include substitution variants, deletion variants and insertion variants. As known in the art, an allelic variant is an alternative form of a polynucleotide. It may be a substitution, deletion or insertion of one or more nucleotides, but the function of encoding the RNA editing enzyme of the present invention will not be substantially changed.

The full-length nucleotide sequence or fragments thereof encoding the fusion protein of the present invention can usually be obtained by PCR amplification method, recombinant method or artificial synthesis method. For the PCR amplification method, primers can be designed according to the published relevant nucleotide sequence, especially the open reading frame sequence, and using a commercially available cDNA library or a cDNA library prepared according to a conventional method known to those skilled in the art as a template, amplifying the relevant sequence. When the sequence is long, it is often necessary to perform two or more PCR amplifications, and then the amplified fragments are spliced together in the correct order.

Once the relevant sequence is obtained, the recombination method can be used to obtain the relevant sequence in large quantities. This is usually done by cloning it into a vector, then transferring it into a cell, and then isolating the relevant sequence from the proliferated host cell by conventional methods.

In addition, artificial synthesis methods can also be used to synthesize related sequences, especially when the fragment length is short. Usually, by first synthesizing multiple small fragments, and then ligating to obtain fragments with very long sequences.

The method of using PCR technology to amplify DNA/RNA is preferably used to obtain the gene of the present invention. The primers used for PCR can be appropriately selected according to the sequence information of the present invention disclosed herein and can be synthesized by conventional methods. The amplified DNA/RNA fragments can be separated and purified by conventional methods such as gel electrophoresis.

The present invention also relates to a vector containing the polynucleotide of the present invention, a host cell produced by genetic engineering using the vector or protein coding sequence of the present invention, and a method for expressing the RNA editing enzyme of the present invention on the NK cell by recombinant technology.

Through conventional recombinant DNA technology, the polynucleotide sequence of the present invention can be used to obtain NK cells expressing the RNA editing enzyme of the present invention. Generally, it includes the steps of: transducing the first expression cassette and/or the second expression cassette of the present invention into NK cells, so as to obtain the NK cells.

Methods well known to those skilled in the art can be used to construct an expression vector containing the coding DNA sequence of the RNA editing enzyme of the present invention and appropriate transcription/translation control signals. These methods include in vitro recombinant DNA technology, DNA synthesis technology, and in vivo recombination technology. The DNA sequence can be effectively linked to an appropriate promoter in the expression vector to guide mRNA synthesis. The expression vector also includes a ribosome binding site for translation initiation and a transcription terminator.

In addition, the expression vector preferably contains one or more selective marker genes to provide phenotypic traits for selection of transformed host cells, such as dihydrofolate reductase for eukaryotic cell culture, neomycin resistance, and green Fluorescent protein (GFP), or tetracycline or ampicillin resistance for E. coli.

A vector containing the above-mentioned appropriate DNA sequence and an appropriate promoter or control sequence can be used to transform an appropriate host cell so that it can express the protein.

The host cell can be a prokaryotic cell, such as a bacterial cell; or a lower eukaryotic cell, such as a yeast cell; or a higher eukaryotic cell, such as a mammalian cell. Representative examples include: Escherichia coli, Bacillus subtilis, Streptomyces bacterial cells; fungal cells such as Pichia pastoris, Saccharomyces cerevisiae cells; plant cells; Drosophila S2 or Sf9 insect cells; CHO, NS0, COST, or 293 cells of animal cells and so on.

Transformation of host cells with recombinant DNA can be carried out by conventional techniques well known to those skilled in the art. When the host is a prokaryotic organism such as Escherichia coli, competent cells that can absorb DNA can be harvested after the exponential growth phase and treated with the CaCl₂ method. The steps used are well known in the art. Another method is to use MgCl₂. If necessary, the transformation can also be carried out by electroporation. When the host is a eukaryote, the following DNA transfection methods can be selected: calcium phosphate co-precipitation method, conventional mechanical methods such as microinjection, electroporation, liposome packaging, etc.

The obtained transformants can be cultured by conventional methods to express the protein encoded by the gene of the present invention. Depending on the host cell used, the medium used in the culture can be selected from various conventional mediums. The culture is carried out under conditions suitable for the growth of the host cell. After the host cells have grown to an appropriate cell density, the selected promoter is induced by a suitable method (such as temperature conversion or chemical induction), and the cells are cultured for a period of time.

The protein in the above method can be expressed in the cell or on the cell membrane, or secreted out of the cell. If necessary, the physical, chemical, and other properties can be used to separate and purify the protein through various separation methods. These methods are well known to those skilled in the art. Examples of these methods include, but are not limited to: conventional renaturation treatment, treatment with a protein precipitation agent (salting out method), centrifugation, bacteria broken through osmosis, ultra-treatment, ultra-centrifugation, molecular sieve chromatography (gel filtration), adsorption chromatography, ion exchange chromatography, high performance liquid chromatography (HPLC) and various other liquid chromatography techniques and combinations of these methods.

Vector

The present invention also provides a vector containing the polynucleotide of the present invention, Vectors derived from retroviruses such as lentiviruses are suitable tools to achieve long-term gene transfer because they allow long-term, stable integration of the transgene and its propagation in daughter cells. Lentiviral vectors have advantages over vectors derived from oncogenic retroviruses such as murine leukemia virus because they can transduce non-proliferating cells, such as hepatocytes. They also have the advantage of low immunogenicity.

In a simple summary, usually by operably linking the expression cassette or nucleic acid sequence of the present invention to a promoter and incorporating it into an expression vector. The vector is suitable for replication and integration of eukaryotic cells. A typical cloning vector contains transcription and translation terminators, initial sequences, and promoters that can be used to regulate the expression of the desired nucleic acid sequence.

The expression construct of the present invention can also use standard gene delivery protocols for nucleic acid immunization and gene therapy. Methods of gene delivery are known in the art. See, for example, U.S. Pat. Nos. 5,399,346, 5,580,859, 5,589,466, which are hereby incorporated by reference in their entirety. In another embodiment, the invention provides a gene therapy vector.

The expression cassette or nucleic acid sequence can be cloned into many types of vectors. For example, the expression cassette or nucleic acid sequence can be cloned into a vector including but not limited to plasmids, phagemids, phage derivatives, animal viruses, and cosmids. Specific vectors of interest include expression vectors, replication vectors, probe generation vectors, and sequencing vectors.

Further, the expression vector can be provided to the cell in the form of a viral vector. Viral vector technology is well known in the art and is described in, for example, Sambrook et al. (2001, Molecular Cloning: A Laboratory Manual, Cold Spring Harbor Laboratory, New York) and other virology and molecular biology manuals. Viruses that can be used as vectors include, but are not limited to, retrovirus, adenovirus, adeno-associated virus, herpes virus, and lentivirus. Generally, a suitable vector contains an origin of replication that functions in at least one organism, a promoter sequence, convenient restriction enzyme sites, and one or more selective markers (e.g., WO01/96584; WO01/29058; and U.S. Pat. No. 6,326,193).

Many virus-based systems have been developed for gene transfer into mammalian cells. For example, retroviruses provide a convenient platform for gene delivery systems. The selected gene can be inserted into a vector and packaged into retroviral particles using techniques known in the art. The recombinant virus can then be isolated and delivered to target cells in vivo or ex vivo. Many retroviral systems are known in the art.

Additional promoter elements, such as enhancers, can regulate the frequency of transcription initiation. Generally, these are located in the 30-110 bp region upstream of the initiation site, although it has recently been shown that many promoters also contain functional elements downstream of the initiation site. The spacing between promoter elements is often flexible in order to maintain promoter function when the elements are inverted or moved relative to one another. In the thymidine kinase (tk) promoter, the spacing between promoter elements can be increased by 50 bp before the activity begins to decrease. Depending on the promoter, it appears that individual elements can act cooperatively or independently to initiate transcription.

An example of a suitable promoter is the early cytomegalovirus (CMV) promoter sequence. The promoter sequence is a strong constitutive promoter sequence capable of driving high-level expression of any polynucleotide sequence operably linked to it. Another example of a suitable promoter is elongation growth factor-1α (EF-1α). However, other constitutive promoter sequences can also be used, including but not limited to the simian virus 40 (SV40) early promoter, mouse breast cancer virus (MMTV), human immunodeficiency virus (HIV) long terminal repeat (LTR) promoter, MoMuLV promoter, avian leukemia virus promoter, Epstein-Barr virus immediate early promoter, Russ sarcoma virus promoter, and human gene promoters, such as but not limited to actin promoter, Myosin promoter, heme promoter and creatine kinase promoter. Further, the present invention should not be limited to the application of constitutive promoters. Inducible promoters are also considered part of the invention. The use of an inducible promoter provides a molecular switch that can turn on expression of a polynucleotide sequence operably linked to an inducible promoter when such expression is desired, or turn off expression when expression is undesirable. Examples of inducible promoters include, but are not limited to, metallothionein promoter, glucocorticoid promoter, progesterone promoter and tetracycline promoter.

The expression vector introduced into the cell may also contain either or both of a selective marker gene or a reporter gene to facilitate the identification and selection of the expression cell from the cell population seeking to be transfected or infected by the viral vector. In other aspects, the selective marker can be carried on a single piece of DNA and used in the co-transfection procedure. Both the selective marker and the reporter gene can be flanked by appropriate regulatory sequences so that they can be expressed in the host cell. Useful selective markers include, for example, antibiotic resistance genes such as neo and the like.

Reporter genes are used to identify potentially transfected cells and to evaluate the functionality of regulatory sequences. Generally, a reporter gene is a gene that does not exist in the recipient organism or tissue or is expressed by the recipient organism or tissue, and it encodes a polypeptide whose expression is clearly indicated by some easily detectable properties such as enzyme activity. After the DNA has been introduced into the recipient cell, the expression of the reporter gene is measured at an appropriate time. Suitable reporter genes may include genes encoding luciferase, β-galactosidase, chloramphenicol acetyltransferase, secreted alkaline phosphatase or green fluorescent protein genes (e.g., Ui-Tei et al., 2000FEBS Letters 479: 79-82).

Physical methods for introducing polynucleotides into host cells include calcium phosphate precipitation, lipofection, particle bombardment, microinjection, electroporation, and so on. Methods of producing cells including vectors and/or exogenous nucleic acids are well known in the art. See, for example, Sambrook et al. (2001, Molecular Cloning: A Laboratory Manual, Cold Spring Harbor Laboratory, New York). The preferred method for introducing polynucleotides into host cells is calcium phosphate transfection.

Biological methods for introducing polynucleotides into host cells include the use of DNA and RNA vectors. Viral vectors, especially retroviral vectors, have become the most widely used method of inserting genes into mammalian, such as human cells. Other viral vectors can be derived from lentivirus, poxvirus, herpes simplex virus I, adenovirus, adeno-associated virus, and so on. See, for example, U.S. Pat. Nos. 5,350,674 and 5,585,362.

Chemical means for introducing polynucleotides into host cells include colloidal dispersion systems, such as macromolecular complexes, nanocapsules, microspheres, and beads; and lipid-based systems, including oil-in-water emulsions, micelles, mixed micelles, and lipidosome. Exemplary colloidal systems used as delivery vehicles in vitro and in vivo are liposomes (e.g., artificial membrane vesicles).

Where a non-viral delivery system is used, an exemplary delivery vehicle is a liposome. Consider using lipid formulations to introduce nucleic acids into host cells (in vitro, ex vivo, or in vivo). In another aspect, the nucleic acid can be associated with lipids. Lipid-associated nucleic acids can be encapsulated in the aqueous interior of liposomes, dispersed in the lipid bilayer of liposomes, and attached to the liposome via a linking molecule associated with both the liposome and the oligonucleotide, trapped in liposomes, complexed with liposomes, dispersed in a solution containing lipids, mixed with lipids, combined with lipids, contained in lipids as a suspension, contained in micelles or complexed with micelles, or otherwise associated with lipids. The lipid, lipid/DNA or lipid/expression vector associated with the composition is not limited to any specific structure in the solution. For example, they may exist in a bilayer structure, as micelles or have a “collapsed” structure. They can also simply be dispersed in the solution, possibly forming aggregates of uneven size or shape. Lipids are fatty substances, which can be naturally occurring or synthetic lipids. For example, lipids include fat droplets, which occur naturally in the cytoplasm and in such compounds containing long-chain aliphatic hydrocarbons and their derivatives such as fatty acids, alcohols, amines, amino alcohols, and aldehydes.

Preparation

The present invention provides the preparation according to the fifth aspect of the present invention. In one embodiment, the preparation is a liquid formulation. Preferably, the preparation is an injection.

In one embodiment, the preparation may include buffers such as neutral buffered saline, sulfate buffered saline, etc.; carbohydrates such as glucose, mannose, sucrose or dextran, mannitol; protein; polypeptides or amino acids such as glycine; Antioxidant; Chelating agent such as EDTA or glutathione; Adjuvant (for example, aluminum hydroxide); and Preservative.

The technical solution of the present invention has the following beneficial effects:

(1) The RNA editing enzyme of the present invention is a single-component protease that does not contain any RNA components and is composed of endogenous human protein sequences. Therefore, these engineered proteins have lower immunogenicity and system complexity than the CRISPR-Cas system in gene therapy. Moreover, the system is more flexible and safer than DNA editing.

(2) The RNA editing enzyme of the present invention has a low off-target rate and high editing efficiency.

(3) The RNA editing enzyme of the present invention has high editing precision and can realize single-base gene editing.

(4) The RNA editing enzyme of the present invention is a protease, which can be targeted to the organelle or nucleus to function through organelle localization signals, for example, a nuclear localization signal NLS is connected to the N-terminal or C-terminal of the RNA editing enzyme, localize the editing enzyme to the nucleus to function, or connect a mitochondrial localization signal MLS to localize the enzyme to the mitochondria to play an editing function.

The present invention will be further explained below in conjunction with specific implementations. It should be understood that these embodiments are only used to illustrate the present invention and not to limit the scope of the present invention. The experimental methods without specific conditions in the following examples are usually based on conventional conditions, such as the conditions described in Sambrook et al., Molecular Cloning: Laboratory Manual (New York: Cold Spring Harbor Laboratory Press, 1989), or according to manufacturing The conditions suggested by the manufacturer. Unless otherwise stated, percentages and parts are calculated by weight.

### EXPERIMENTAL METHOD

The deaminated catalytic domains of members of the RNA adenosine deaminase ADAR family and members of the RNA cytidine deaminase APOBEC family were cloned and fused with the RNA binding protein PUF to form two different RNA editing enzymes PARSE and APRSE. According to the above design, it was verified that PARSE and APRSE RNA editing enzymes can edit RNA at the cellular level. Detecting the editing efficiency and precision of these enzymes, and analyzing the off-target effects of these enzymes at the transcriptome level. The constructed RNA editing enzyme is used to repair some single-base pathogenic point mutations, which are used to repair the pathogenic point mutations of GRIA2, COL3A1, DMD, EZH2, SCN1A and SCN5A genes.

Specifically, it includes the following steps:

1. Based on the existing RNA binding protein PUF, the dADAR, ADAR1 and ADAR2 genes encoding the ADAR protein were cloned separately using molecular cloning technology, and then these two genes were spliced together to form a new fusion gene. The PARSE protein was encoded by the new fusion gene, constructing and forming different A-to-I RNA site-directed editing enzymes. Using molecular cloning technology to fuse different RNA cytidine deaminase (human apobec1, human apobec3a, human3b, human3c, human3d, human3g, human3h, humanAID, mouse APOBEC1, mouse APOBEC3A, mouse AID, Rat APOBEC1) to PUF, constructing and forming different C-to-U RNA site-directed editing enzymes.

2. Detecting the editing activity of these new RNA editing enzymes at the cellular level, transferring the PARSE RNA editing enzyme and the GFP reporter gene plasmid into the cells at the same time, and after 48 hours, the RNA of the GFP reporter gene was collected and sequenced to detect RNA editing events.

3. Detecting the editing activity of these new RNA editing enzymes at the cellular level, transferring the APRSE RNA editing enzyme and the GFP reporter gene plasmid into the cells at the same time, and after 48 hours, the RNA of the GFP reporter gene was collected and sequenced to detect RNA editing events.

4. Detecting and analyzing the efficiency, precision and off-target rate of RNA editing enzymes through RNA seq high-throughput sequencing technology.

5. Using the constructed RNA editing enzyme to repair some single-base disease-causing mutations, and using PARSE to repair the pathogenic point mutations such as GRIA2, COL3A1 and DMD.

6. Using the constructed RNA editing enzyme to repair some single-base disease-causing mutations, and using APRSE to repair the pathogenic point mutations of genes such as EZH2, SCN1A and SCN5A.

### Example 1

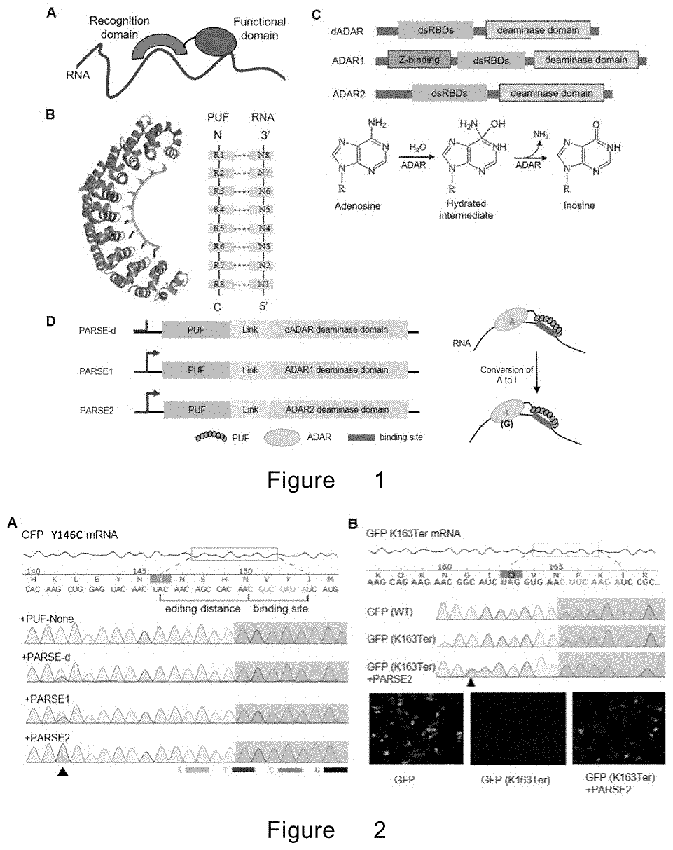

ADAR is an adenosine editing enzyme that can catalyze the deamination of RNA adenosine to form inosine (adenosine-to-inosine, A-to-I). The ADAR catalytic domains from three different sources were cloned and fused with the RNA binding protein PUF through linker, and three different artificial RNA editing enzymes (RNA editase) were developed and the system was named artificial PUF-ADAR RNA sequence editors (PARSE-d, PARSE1, PARSE2) (FIG. 1, A-D)

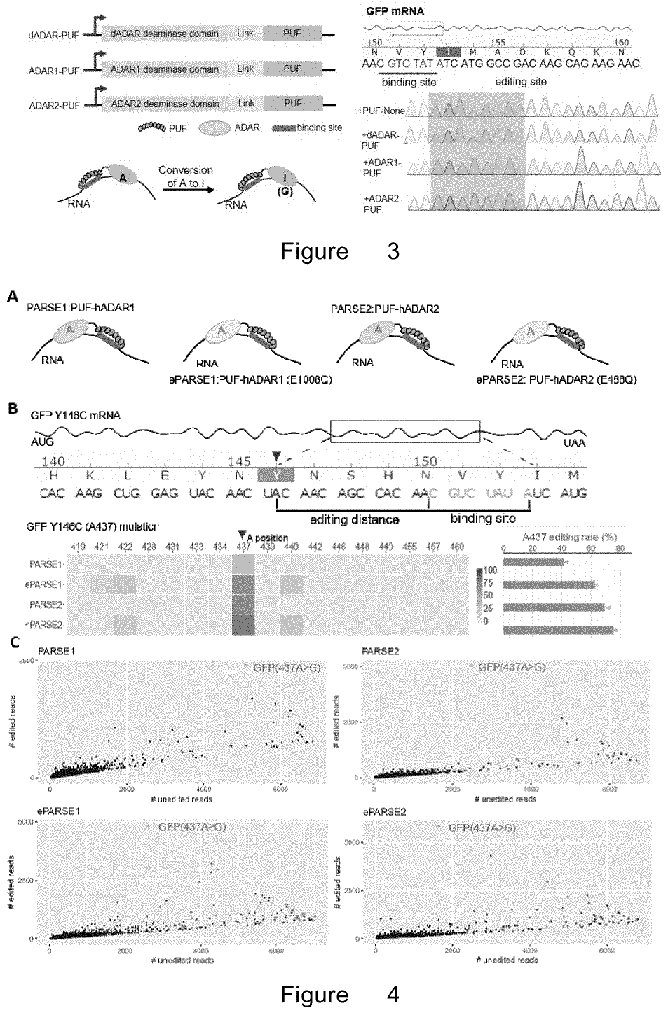

Detecting the editing activity of the three RNA editing enzymes, PARSE-d, PARSE1 and PARSE2 at the cell level, transferring the PARSE RNA editing enzyme and the GFP reporter gene plasmid into the cells at the same time, and after 48h, the RNA of the GFP reporter gene was collected and sequenced to detect RNA editing events, proving that PARSE-d, PARSE1 and PARSE2 have the ability to edit target RNA efficiently (FIG. 2A). By constructing a GFP mRNA containing an early stop codon, editing from A to G at a specific site through PARSE, thereby restoring the expression of the GFP gene, proving that PARSE has high-precision fixed-point editing capabilities (FIG. 2B).

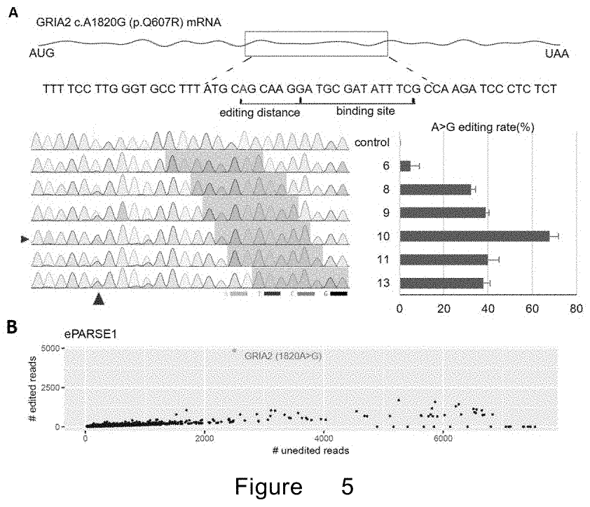

The ADAR catalytic domains from three different sources were cloned, and fused through linker with RNA binding protein PUF, ADAR was placed at the N end of the newly designed fusion protein, and PUF was placed at the C end of the newly designed fusion protein to form a new RNA editing enzyme with new ADAR catalytic domain first and PUF RNA binding protein behind, using this newly synthesized RNA editing enzyme to edit target RNA, and no editing events were detected (FIG. 3). This proves that ADAR-dependent RNA editing enzymes have strict requirements on the position of RNA binding proteins, and the RNA editing enzyme of the present invention has excellent RNA editing activity.

### Example 2

By optimizing the ADAR catalytic domain to improve the editing efficiency of ADAR on target RNA, and through the RNA seq high-throughput sequencing technology, the efficiency, accuracy and off-target rate of RNA editing enzymes can be detected and analyzed. A new point mutation was introduced into the catalytic domains of ADAR1 and ADAR2 to improve the editing efficiency of ADAR (FIG. 4A). The RNA seq high-throughput sequencing technology was used to detect and analyze the efficiency and accuracy of RNA editing enzymes. It has been found that PARSE1, ePARSE1, PARSE2, and ePARSE2 can all edit target RNA with efficiencies of 42%, 65%, 67%, and 78%, respectively (FIG. 4B). The analysis of RNA-seq results shows that PARSE editing has a certain degree of off-target rate, but compared with the editing efficiency of the target site, the off-target efficiency is lower. The off-target rate can be reduced by reducing the amount of PARSE transfection in the later optimization process (FIG. 4C).

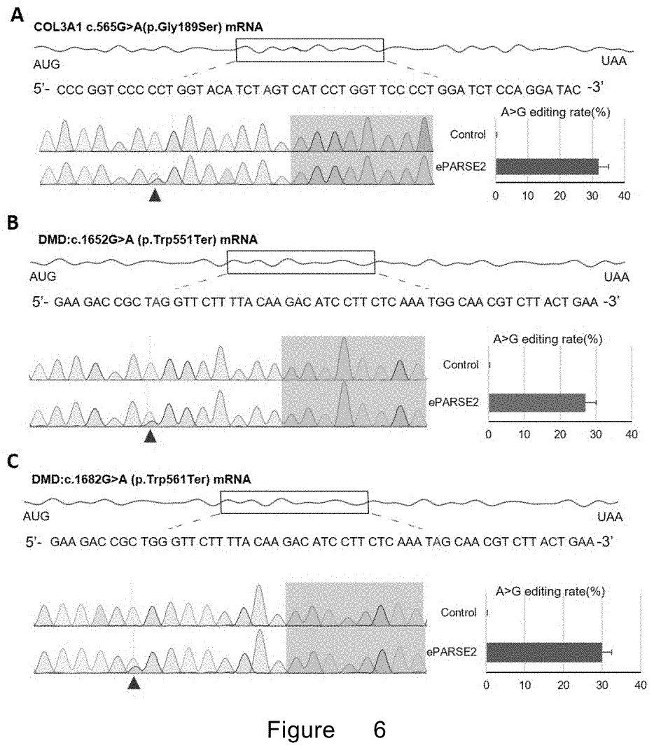

Using the constructed ePARSE1 to repair the pathogenic editing site of GRIA2 gene. GRIA2 is a subunit of calcium channel protein. The mutation of the position 607 amino acid of the protein will cause the calcium channel protein to be unable to close, resulting in a pathogenic phenotype. Using ePARSE1 to perform site-specific repair on this site, the results show that the repair efficiency is 68%, that is, the repair of the pathogenic point mutation of GRIA2 (p.Q607R) can be completed at the cellular level, providing a powerful tool for further treatment of this gene mutation (FIG. 5A), and the off-target efficiency of this site is low (FIG. 5B), showing a very good application prospect of ePARSE1 to treat this disease.

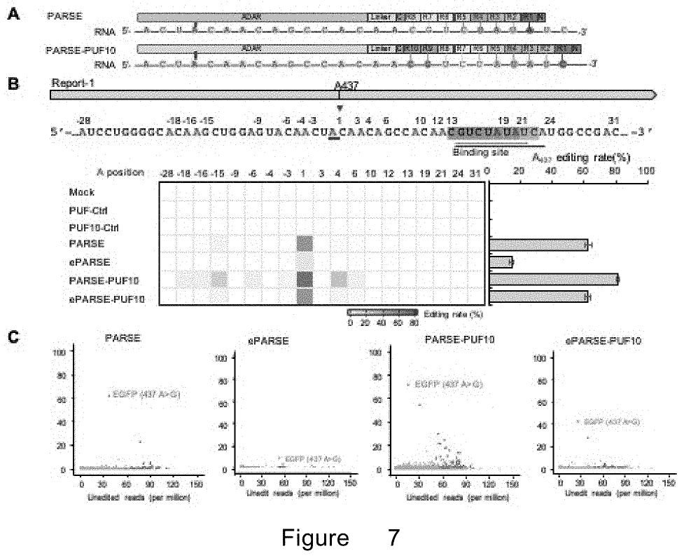

The constructed ePARSE2 was used for site-specific repair of the pathogenic point mutations of the COL3A1 and DMD genes. The results show that the repair efficiency is 33%, 25%, and 30%, respectively, that is, the site-specific repair of the pathogenic point mutations of COL3A1 and DMD genes can be completed at the cellular level, providing a powerful tool for further treatment of this gene mutation (FIG. 6A, 6B, 6C), showing the good application prospects of ePARSE2 in the treatment of this type of disease.

### Example 3

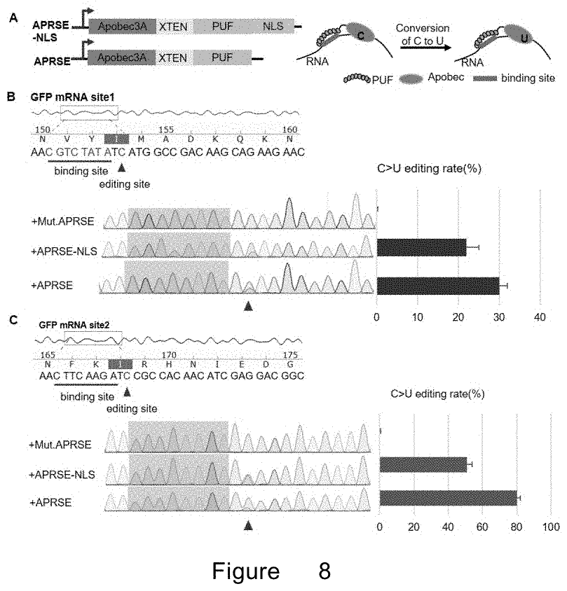

Optimize the RNA binding protein PUF to improve the accuracy of PARSE editing RNA and reduce the off-target rate. By optimizing the PUF domain, the PUF8 that recognizes 8 bases was optimized to the PUF10 that recognizes and binds to ten bases (FIG. 7, A-C), this optimized design does not reduce the editing efficiency of RNA target sites, and as the number of PUF recognition and binding bases is increased, this strategy reduces the off-target rate of PARSE by 10 times, effectively reducing off-target effects and has the potential for further optimization, PUF can be optimized to recognize and bind 12 bases, 16 bases, or even longer, which can greatly expand the application range of PARSE.

### Example 4

APOBECs can catalyze the nucleotide editing of cytidine-to-uridine (C-to-U), and the catalytic domains of RNA cytidine deaminase from various sources of APOBEC family (human apobec1, human apobec3a, human3b, human3c, human3d, human3g, human3h, humanAID, mouse APOBEC1, mouse APOBEC3A, mouse AID, Rat APOBEC1) were cloned and fused with the RNA binding protein PUF through linker to form a variety of new C to U RNA site-directed editing enzymes, the system is named artificial APOBEC-PUF RNA sequence editors (APRSE), and is further subdivided into APRSE-NLS that can enter the nucleus and APRSE that is expressed in the cytoplasm. The figure below takes Apobec3A as an example (FIG. 8A).

Detecting the editing activity of the two RNA editing enzymes APRSE-NLS and APRSE at the cell level, transferring the APRSE RNA editing enzyme and the GFP reporter gene plasmid into the cells at the same time, and after 48h, the RNA of the GFP reporter gene was collected and sequenced to detect RNA editing events. It is proved that APRSE-NLS and APRSE have the ability to edit target RNA efficiently (FIG. 8B, 8C). The results show that APRSE has high-precision fixed-point editing ability, which can directly edit the second base downstream of the APRSE binding site with high precision and efficiency, the editing efficiency of different editing sites is between 30% and 80%.

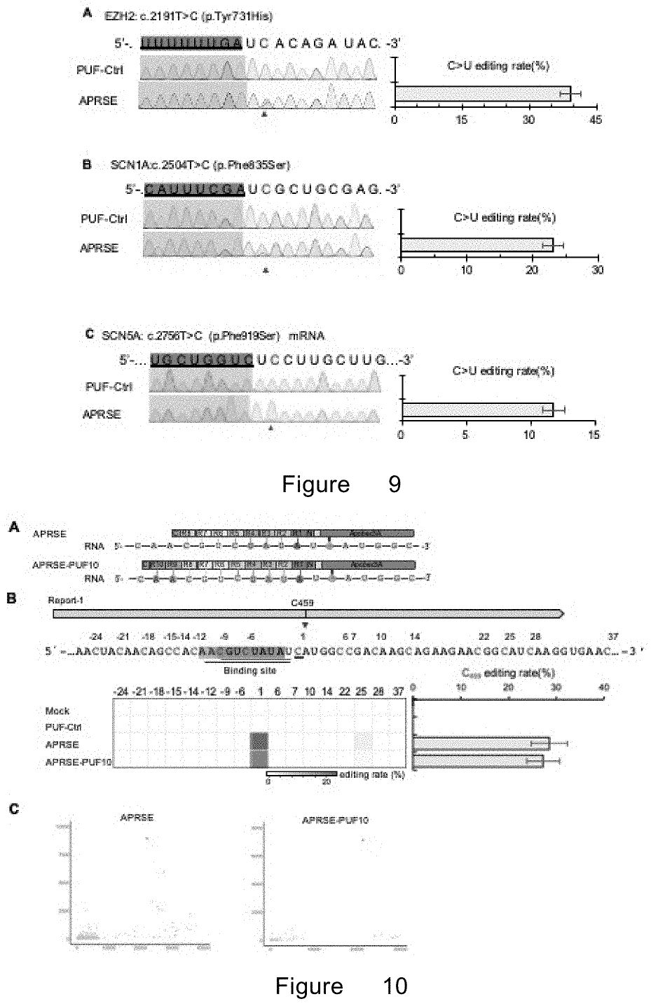

The constructed APRSE was used for site-specific repair of the pathogenic point mutations of EZH2, SCN1A and SCN5A genes. The results show that the repair efficiency is 39%, 23%, and 12% respectively, that is, the site-specific repair of the pathogenic point mutations of EZH2, SCN1A and SCN5A genes can be completed at the cellular level, providing a powerful tool for further treatment of this gene mutation (FIGS. 9A, 9B, 9C), showing the good application prospects of APRSE to treat this type of disease.

### Example 5

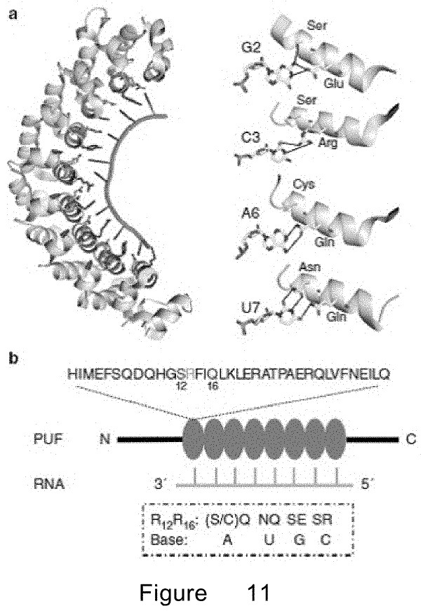

Optimizing the RNA-binding protein PUF to improve the accuracy of APRSE's RNA editing and reduce off-target efficiency. By optimizing the PUF domain, the PUF8 that recognizes 8 bases is optimized to the PUF10 that recognizes and binds to ten bases (FIG. 10, A-C), this optimized design does not reduce the editing efficiency of RNA target sites, and with increasing the number of PUF recognition and binding bases, this strategy reduces the off-target effect of APRSE by 12 times, effectively reducing off-target effects, and has the potential for further optimization, PUF can be optimized to recognize and bind 12 bases, 16 bases, or even longer, which can greatly expand the application range of APRSE.

### DISCUSSION

Compared with direct specific editing and manipulation of DNA, specific manipulation of RNA is reversible and has greater flexibility. RNA-targeted gene therapy can effectively avoid the shortcomings of DNA gene therapy. Therefore, the manipulation of genes at the RNA level has better controllability and safety, making this type of gene therapy more conducive to the transformation of basic research into clinical practice.

The current base editing at the RNA level mainly includes REPAIR based on CRISPR-Cas13 and the method of recruiting cell endogenous RNA adenosine deaminase ADAR through oligonucleotide fragments for RNA editing. REPAIR based on CRISPR-Cas13 also has the same problems as the above-mentioned DNA editing, including the system is too large, editing efficiency and editing accuracy are low, and it is easy to cause immune responses and other problems; Based on oligonucleotide fragments recruiting cell endogenous RNA adenosine deaminase for RNA editing, the current application range is too small, there are also difficulties in delivery and single efficacy, and the system is more complicated.

The present inventors fused the RNA recognition domain with the functioning utility domain (effector domain) to form a new functional protein, which specifically targets the target RNA through the recognition domain and utilizes the utility domain to perform RNA editing, to eliminate pathogenic RNA by correcting the wrong point mutations in the DNA. The artificially constructed RNA editing enzyme is a single-component protease that does not contain any RNA components and is composed of endogenous human protein sequences. Therefore, these engineered proteins have lower immunogenicity and system complexity than the CRISPR-Cas system in gene therapy. Moreover, the system is more flexible and safer than DNA editing.

In addition, because the artificial RNA editing enzyme based on a single protein can connect different cell location sequences to locate in different subcellular structures (including nucleus, cytoplasm, mitochondria, chloroplasts, etc.), artificial RNA editing enzymes can specifically edit RNA in different subcellular structures. Taking mitochondria as an example, there is currently no effective editing method for mitochondrial genes, so that artificial RNA editing enzymes can have an irreplaceable role in mitochondrial gene manipulation.

Therefore, constructing modular artificial RNA-binding proteins through synthetic biological means, and fusing RNA editing proteins to RNA-binding proteins, so as to specifically control the editing of targeted RNAs, is a new treatment idea for targeted RNAs. Since the artificially designed PUF factor can be reprogrammed to recognize almost any 8-nucleotide sequence, it can theoretically be used to edit any given RNA transcript. It is hoped that through the application of this system, some disease-related mutations can be targeted and edited. This system provides useful tools for targeted editing of RNA in human cells, and hopefully treats some diseases caused by nucleic acid mutations. Moreover, the artificially constructed PUF-Factor is a simple enzyme that does not contain any RNA components. It is composed of endogenous human protein sequences. These engineered proteins may be a simpler and more practical alternative than the CRISPR-Cas system in gene therapy.

All publications mentioned herein are incorporated by reference as if each individual document was cited as a reference, as in the present application. It should also be understood that, after reading the above teachings of the present invention, those skilled in the art can make various changes or modifications, equivalents of which falls in the scope of claims as defined in the appended claims.

1. An RNA editing enzyme, comprising:
(a) a RNA recognition domain, the RNA recognition domain is used to recognize the RNA recognition sequence of the RNA sequence to be edited, and bind to the RNA recognition sequence;
(b) a utility domain, which is used for nucleotide editing of the RNA sequence to be edited;
wherein, the RNA recognition domain and the utility binding domain are operably linked.

2. The RNA editing enzyme of claim 1, wherein the utility domain is selected from the group consisting of the deamination catalytic domain of ADAR family members, the deamination catalytic domain of APOBEC family members, RNA methylase, RNA demethylase, added uracil synthase, and a combination thereof.

3. The RNA editing enzyme of claim 1, wherein the RNA recognition domain contains n recognition units, and each recognition unit is used to recognize an RNA base, wherein n is a positive integer of 5-30.

4. The RNA editing enzyme of claim 1, wherein the RNA editing enzyme further includes one or more elements selected from the group consisting of linker peptide, tag sequence, signal peptide sequence, location peptide sequence, and a combination thereof.

5. The RNA editing enzyme of claim 1, wherein the RNA editing enzyme includes an RNA recognition domain and a utility domain, as well as an optional linker peptide, tag sequence, signal peptide sequence and/or location peptide sequence.

6. The RNA editing enzyme of claim 1, wherein the structure of the RNA editing enzyme is shown in any one of the following Formula I to formula IV:
D-L2-A-L1-B (I);
D-L2-B-L1-A (II);
A-L1-B-L2-D (III);
B-L1-A-L2-D (IV);
wherein each “-” is independently a linker peptide or a peptide bond;
A is a RNA recognition domain;
B is a utility domain;
L1 and L2 is each independently none or a linker peptide;
D is none or a location peptide.

7. The RNA editing enzyme of claim 2, wherein the ADAR family member includes: dADAR, ADAR1, ADAR2, TadA, and a combination thereof.

8. An isolated polynucleotide which encodes the RNA editing enzyme of claim 1.

9. A vector, which comprises the polynucleotide of claim 8.

10. (canceled)

11. A method for editing RNA, wherein the method comprising the steps:
(1) providing RNA to be edited and the RNA editing enzyme of claim 1; and
(2) using the RNA editing enzyme of claim 1 to edit the RNA.

12. A method for treating diseases, wherein the method comprising: administering the RNA editing enzyme of claim 1 to a subject in need.
